(19) United States
(12) Patent Application Publication
(10) Pub. No.: US 2023/0049366 A1
(43) Pub. Date: Feb. 16, 2023

(54) CIRCULATION SYSTEM AND METHOD FOR DETECTING IMMUNE ADHERENCE OF PORCINE ERYTHROCYTES

(71) Applicant: Shanxi Agricultural University, Jinzhong City (CN)

(72) Inventors: Hongquan Li, Jinzhong City (CN); Zhen Hou, Jinzhong City (CN); Wei Yin, Jinzhong City (CN); Kuohai Fan, Jinzhong City (CN); Na Sun, Jinzhong City (CN); Panpan Sun, Jinzhong City (CN); Yaogui Sun, Jinzhong City (CN)

(21) Appl. No.: 17/881,069

(22) Filed: Aug. 4, 2022

(30) Foreign Application Priority Data
Aug. 4, 2021 (CN) 202110892012.X

(51) Int. Cl. G01N 15/14

(52) U.S. Cl. CPC G01N 15/1404; G01N 15/1434; G01N 2015/1006

(57) ABSTRACT

The present disclosure provides circulation system and method for detecting immune adherence of porcine erythrocytes. The system includes: a flow chamber, a gasket, a mobile-phase liquid storage bottle, a peristaltic pump, silicone hoses, and a cell culture dish provided with a glass slide, where the gasket is placed on an edge of the glass slide, and the flow chamber is covered on the gasket; the flow chamber, the mobile-phase liquid storage bottle, and the peristaltic pump are connected in sequence with the silicone hoses to form the circulation system; and a porcine erythrocyte suspension of immune adherence-sensitized GFP-E. coli is injected into the mobile-phase liquid storage bottle. In the present disclosure, the circulation system and method for detecting immune adherence of porcine erythrocytes can be used for detection and analysis of working parameters such as a mobile phase flow velocity, a shear force, and a maximum retention time.

### CROSS REFERENCE TO RELATED APPLICATION

This patent application claims the benefit and priority of Chinese Patent Application No. 202110892012.X, filed on Aug. 4, 2021, the disclosure of which is incorporated by reference herein in its entirety as part of the present application.

### TECHNICAL FIELD

The present disclosure relates to the technical field of immune adherence detection of porcine erythrocytes, in particular to a circulation system and method for detecting immune adherence of porcine erythrocytes.

### BACKGROUND ART

Like leukocytes, erythrocytes in human beings and various animals have important immune functions. The erythrocyte has C3b receptors on a surface, and erythrocytes can play a variety of roles, such as clearing immune complexes, promoting phagocytosis, presenting antigens, and activating complements through the C3b receptors on the surface. Therefore, establishing a method for detecting erythrocyte immune function has become a key technology in the art. At present, there is still a lack of a systematic detection system and the establishment of working parameters in the detection of immune adherence of porcine erythrocytes, which are urgently explored by relevant technical personnel.

### SUMMARY

An objective of the present disclosure is to provide a circulation system and method for detecting immune adherence of porcine erythrocytes, which can be used for detection and analysis of working parameters such as a mobile phase flow velocity, a shear force, and a maximum retention time.

To achieve the above objective, the present disclosure provides the following technical solutions.

The present disclosure provides a circulation system for detecting immune adherence of porcine erythrocytes, including: a flow chamber, a gasket, a mobile-phase liquid storage bottle, a peristaltic pump, silicone hoses, and a cell culture dish provided with a glass slide, where the gasket is placed on an edge of the glass slide, and the flow chamber is covered on the gasket; the flow chamber, the mobile-phase liquid storage bottle, and the peristaltic pump are connected in sequence with the silicone hoses to form the circulation system; and a porcine erythrocyte suspension of immune adherence-sensitized GFP-E. coli is injected into the mobile-phase liquid storage bottle.

Further, 20 mL of the porcine erythrocyte suspension of immune adherence-sensitized GFP-E. coli may be injected.

The present disclosure further provides a circulation method for detecting immune adherence of porcine erythrocytes applied to the circulation system for detecting immune adherence of porcine erythrocytes, including the following steps:

S1, starting the peristaltic pump to make the porcine erythrocyte suspension in the mobile-phase liquid storage bottle flow and fill the entire circulation system;

S2, setting a rotational speed of the peristaltic pump, and recording a volume of the suspension collected by a collection tube at each rotational speed within a period of time;

S3, calculating a flow velocity of the porcine erythrocyte suspension in the circulation system, and repeating detection n times, n≥3; and

S4, calculating a shear force of the porcine erythrocyte suspension flowing at each rotational speed in the circulation system.

Further, in step S3, the flow velocity of the porcine erythrocyte suspension in the circulation system may be calculated by the following formula:

$$Q = \frac{{V}_{n} - {V}_{0}}{120 s} \qquad ( 1 )$$

in the formula, Q represents the flow velocity, in mL/s; Vₙ represents the volume of the suspension collected at each rotational speed within the period of time, and V₀ represents an initial volume.

Further, in step S4, the shear force of the porcine erythrocyte suspension flowing at each rotational speed in the circulation system may be calculated by the following formula:

$$τ = \frac{6 μμ}{{a}^{2} b} \qquad ( 2 )$$

in the formula, τ is the shear force, in dynes/cm²; μ is a medium viscosity of 0.0076 P; a is a channel height of 0.013 cm of the flow chamber; b is a channel width of 1.0 cm; and Q is the flow velocity, in mL/s.

Further, the period of time may be set to 120 sec.

Further, the method may further include determining a maximum retention time of a mobile phase:

after the porcine erythrocyte suspension flows and fills the entire circulation system, setting the rotational speed of the peristaltic pump;

pipetting a 0.2 mL sample of the porcine erythrocyte suspension from the mobile-phase liquid storage bottle at regular intervals, and detecting a surface fluorescence intensity of the porcine erythrocytes at each time point by a flow cytometer, where 10⁵ of the porcine erythrocytes are detected in each tube and the samples at each time point are detected 3 times; and calculating an average fluorescence intensity at each time point; and

calculating differences between fluorescence intensities at different time points by one-way ANOVA.

According to a specific example, the present disclosure provides the following technical effects: in the circulation system and method for detecting immune adherence of porcine erythrocytes, the flow chamber, the gasket, the mobile-phase liquid storage bottle, the peristaltic pump, the silicone hoses, and the cell culture dish provided with a glass slide constitutes a cyclic detection system, which can be used for determination of the mobile phase flow velocity, the shear force, and the maximum retention time. The present disclosure provides reliable working parameters for detecting immune adherence of porcine erythrocytes, which is convenient for technicians to operate and conduct follow-up research.

### BRIEF DESCRIPTION OF THE DRAWINGS

To describe the embodiments of the present disclosure or the technical solutions in the related art more clearly, the accompanying drawings required in the embodiments are briefly introduced below. Obviously, the accompanying drawings described below are only some embodiments of the present disclosure. A person of ordinary skill in the art may further obtain other accompanying drawings based on these accompanying drawings without creative labor.

FIG. 1 shows a schematic diagram of changes in a surface fluorescence intensity of porcine erythrocytes at different time points in an example of the present disclosure;

FIG. 2A shows a representative chart of the surface fluorescence intensity of porcine erythrocytes at a first time in the example of the present disclosure;

FIG. 2B shows a representative chart of the surface fluorescence intensity of porcine erythrocytes at another time in the example of the present disclosure;

FIG. 2C shows a representative chart of the surface fluorescence intensity of porcine erythrocytes at another time in the example of the present disclosure;

FIG. 2D shows a representative chart of the surface fluorescence intensity of porcine erythrocytes at another time in the example of the present disclosure;

FIG. 2E shows a representative chart of the surface fluorescence intensity of porcine erythrocytes at another time in the example of the present disclosure;

FIG. 2F shows a representative chart of the surface fluorescence intensity of porcine erythrocytes at another time in the example of the present disclosure;

FIG. 2G shows a representative chart of the surface fluorescence intensity of porcine erythrocytes at another time in the example of the present disclosure;

FIG. 2H shows a representative chart of the surface fluorescence intensity of porcine erythrocytes at another time in the example of the present disclosure;

FIG. 2I shows a representative chart of the surface fluorescence intensity of porcine erythrocytes at another time in the example of the present disclosure;

FIG. 2J shows a representative chart of the surface fluorescence intensity of porcine erythrocytes at another time in the example of the present disclosure;

FIG. 2K shows a representative chart of the surface fluorescence intensity of porcine erythrocytes at another time in the example of the present disclosure;

FIG. 2L shows a representative chart of the surface fluorescence intensity of porcine erythrocytes at another time in the example of the present disclosure;

FIG. 2M shows a representative chart of the surface fluorescence intensity of porcine erythrocytes at another time in the example of the present disclosure; and

FIG. 3 shows a flow chart of the circulation method for detecting immune adherence of porcine erythrocytes according to the present disclosure.

### DETAILED DESCRIPTION OF THE EMBODIMENTS

The technical solutions of the embodiments of the present disclosure are clearly and completely described below with reference to the accompanying drawings. The described embodiments are merely a part rather than all of the embodiments of the present disclosure. All other embodiments obtained by those of ordinary skill in the art based on the embodiments of the present disclosure without creative efforts or undue experimentation shall fall within the protection scope of the present disclosure.

An objective of the present disclosure is to provide circulation systems and methods for detecting immune adherence of porcine erythrocytes, which can be used for detection and analysis of working parameters such as a mobile phase flow velocity, a shear force, and a maximum retention time.

To make the above-mentioned objective, features, and advantages of the present disclosure clearer and more comprehensible, the present disclosure will be further described in detail below in conjunction with the accompanying drawings and specific embodiments.

In an example, the present disclosure provides a circulation system for detecting immune adherence of porcine erythrocytes, including: a flow chamber, a gasket, a mobile-phase liquid storage bottle, a peristaltic pump, silicone hoses, and a cell culture dish provided with a glass slide, where the gasket can be placed on an edge of the glass slide, and the flow chamber can be covered on the gasket; the flow chamber, the mobile-phase liquid storage bottle, and the peristaltic pump can be connected in sequence with the silicone hoses to form the circulation system; and 20 mL of a porcine erythrocyte suspension of immune adherence-sensitized GFP-E. coli can be injected into the mobile-phase liquid storage bottle. Data for an implementation of this circulation system are also provided.

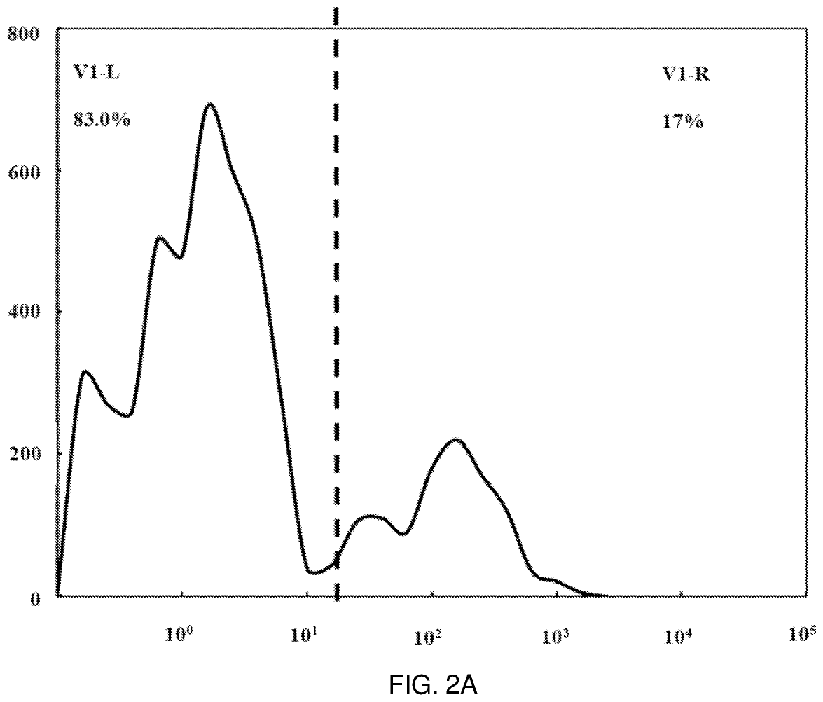

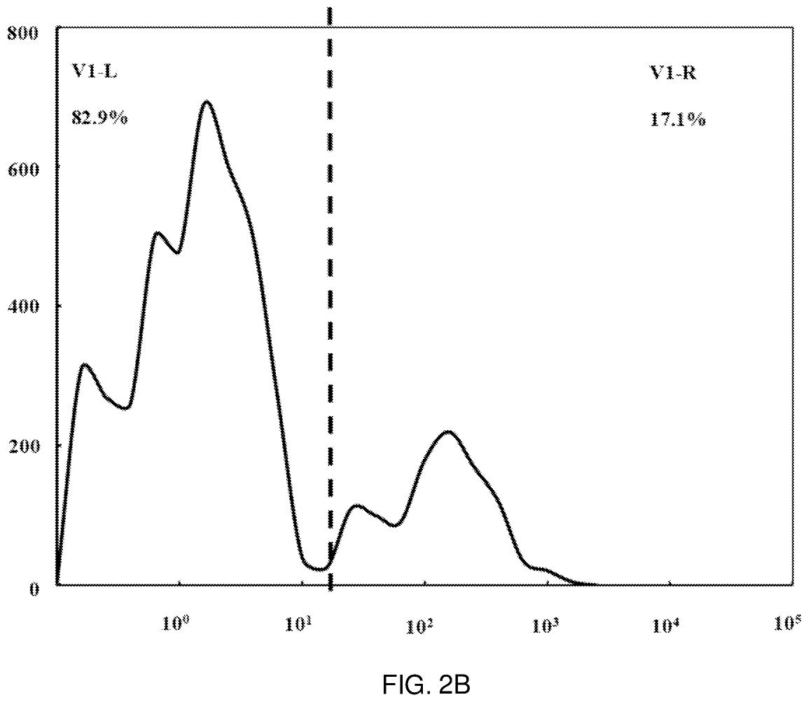

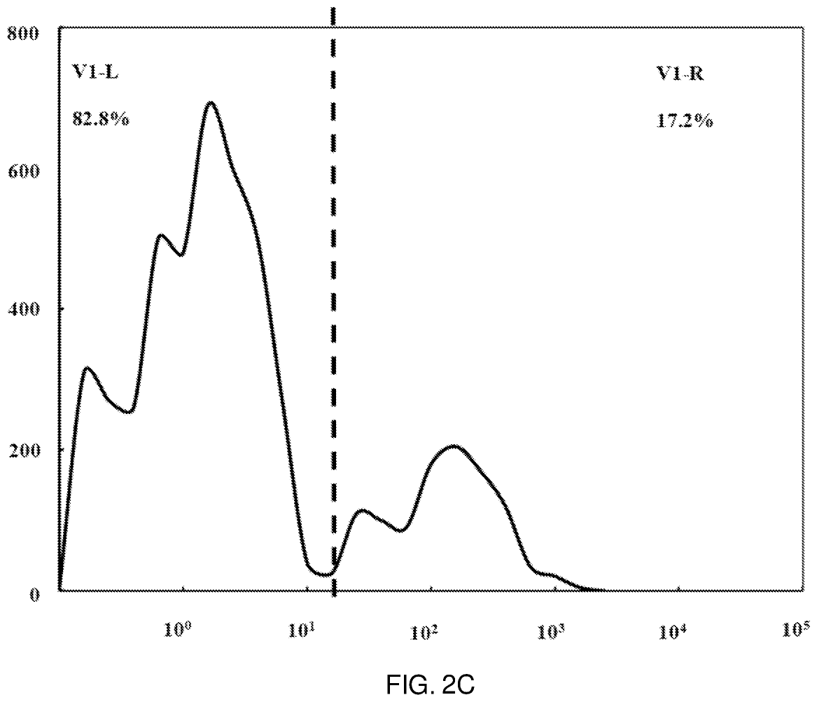

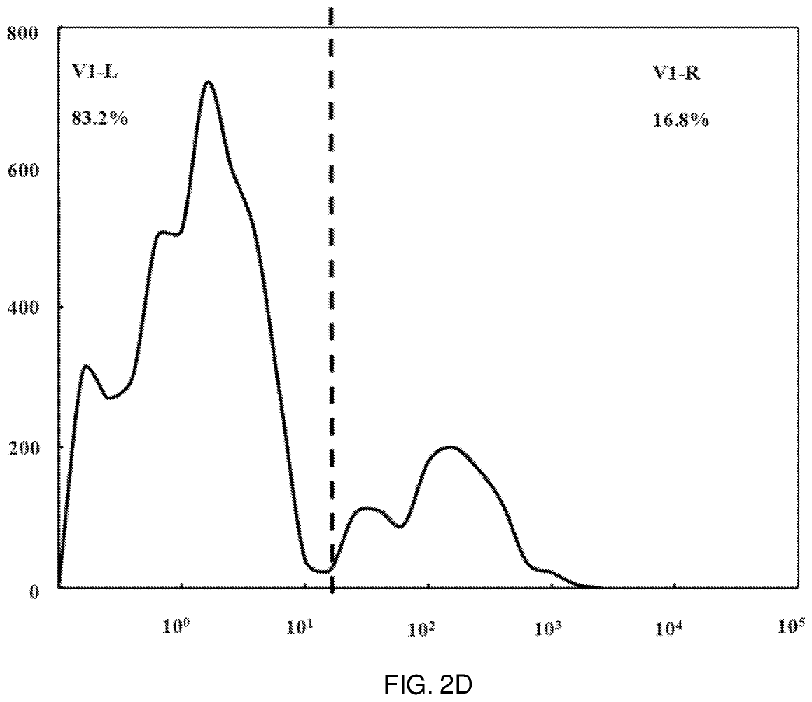

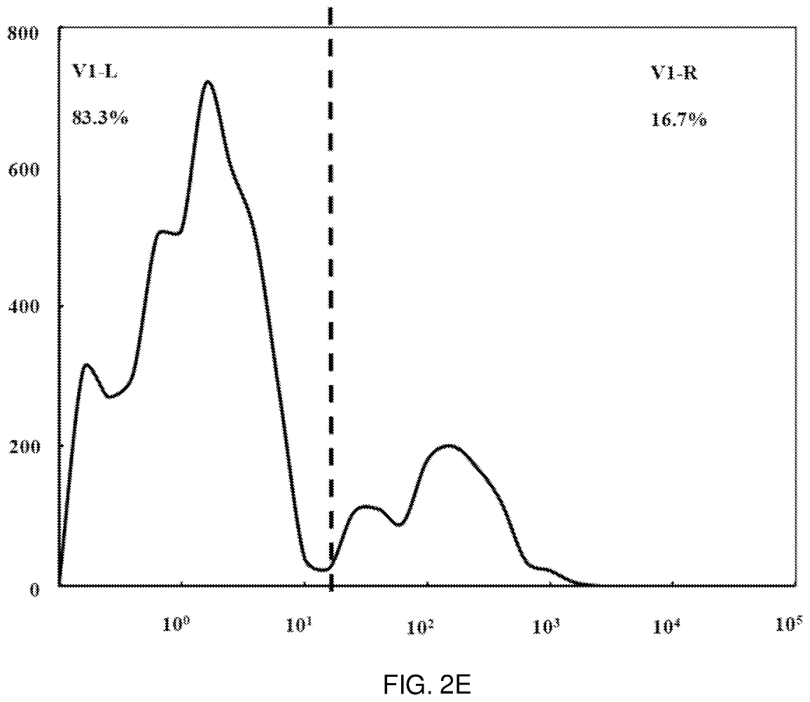

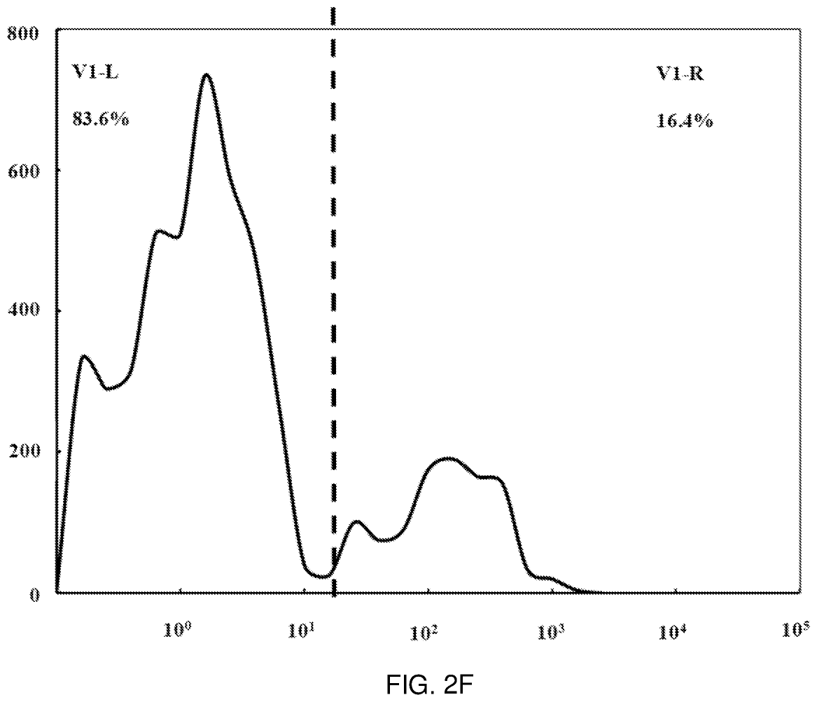

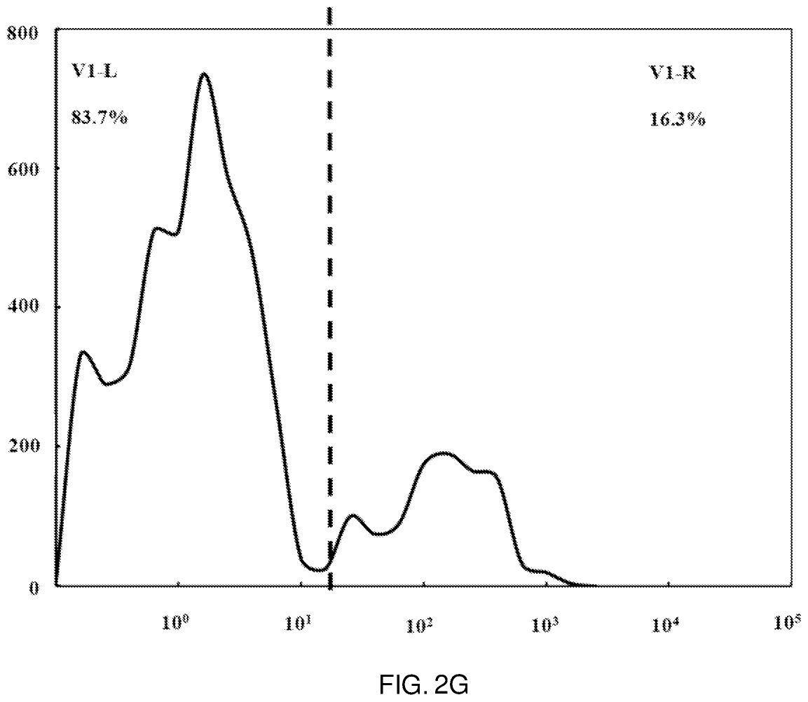

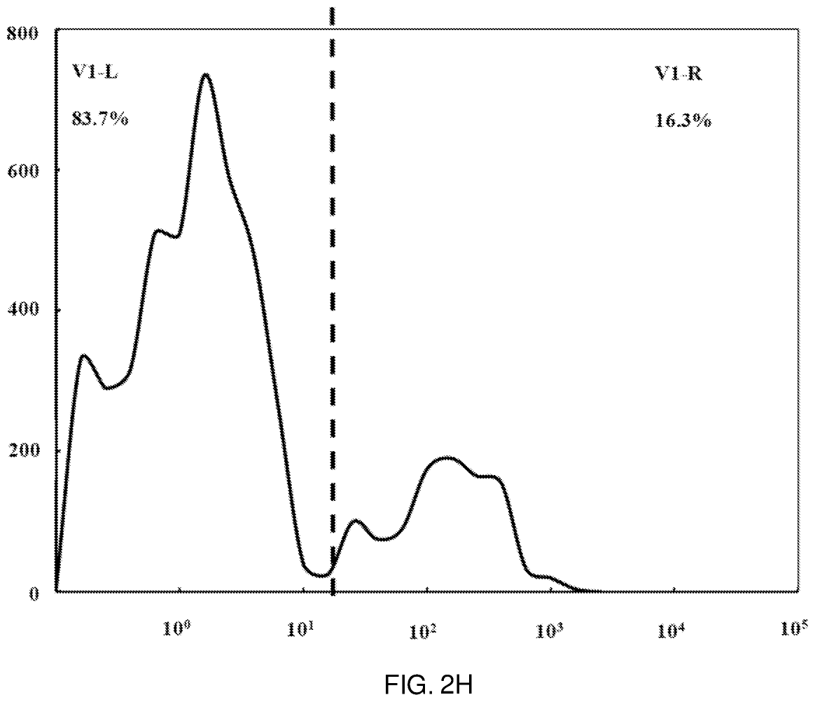

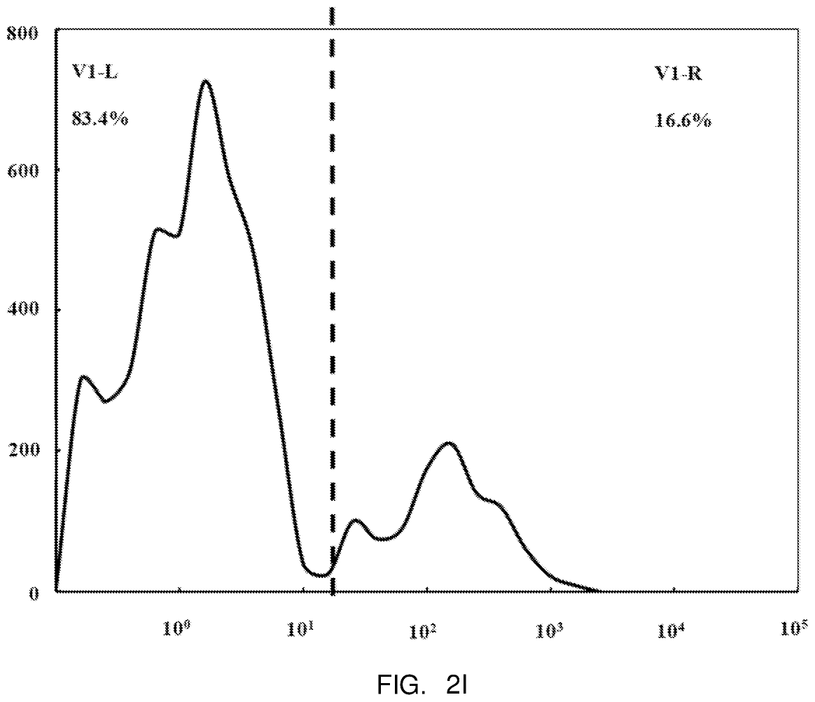

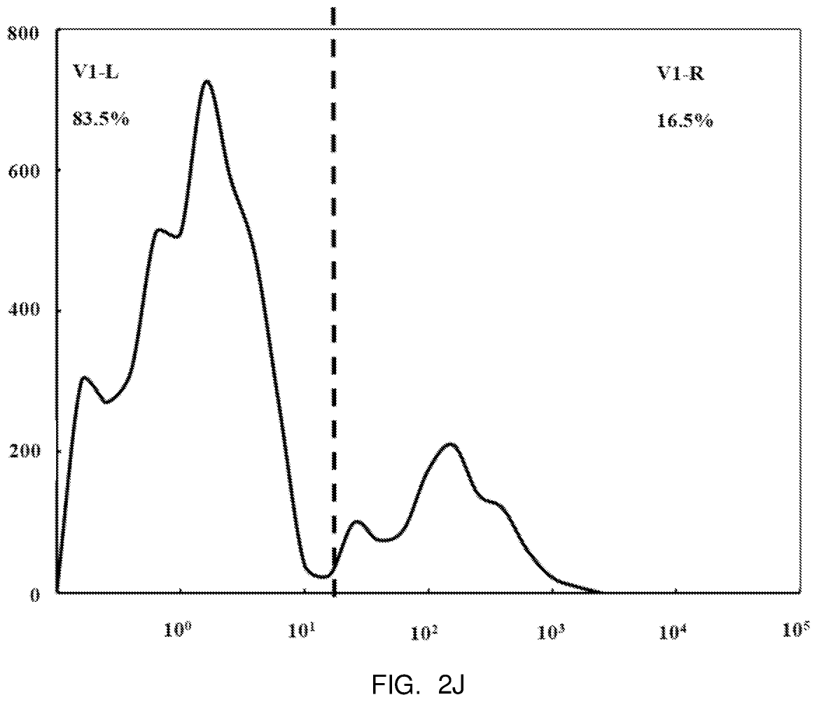

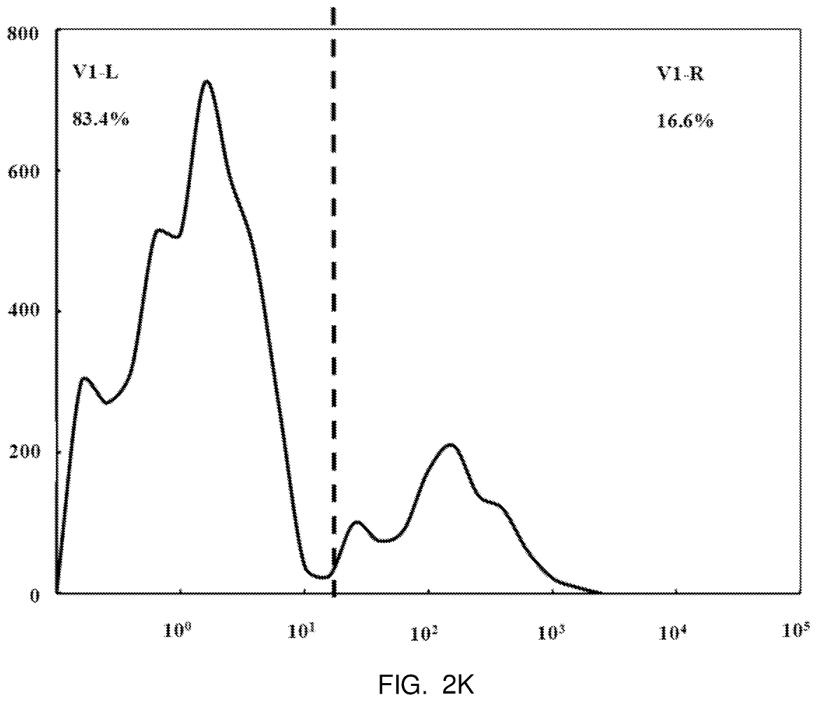

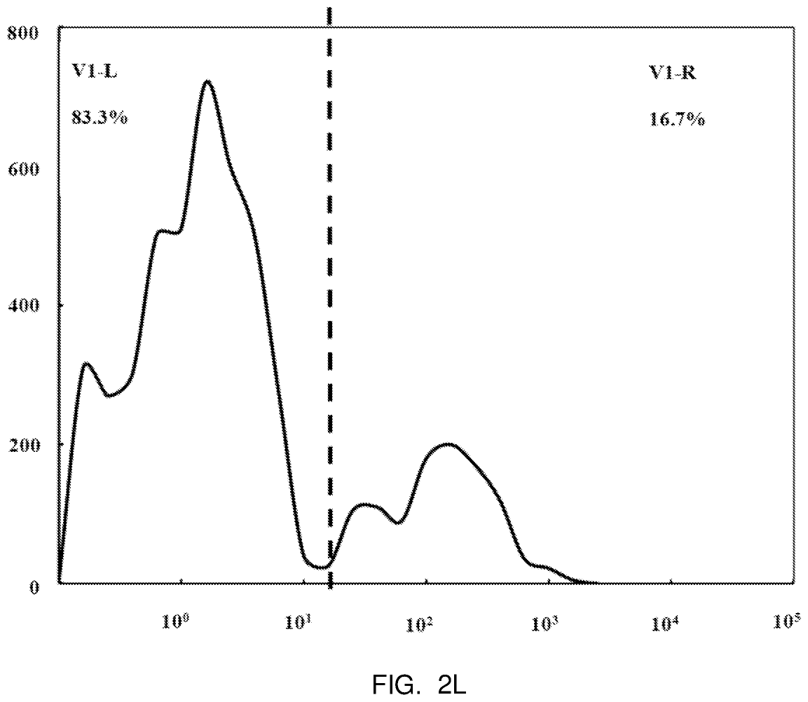

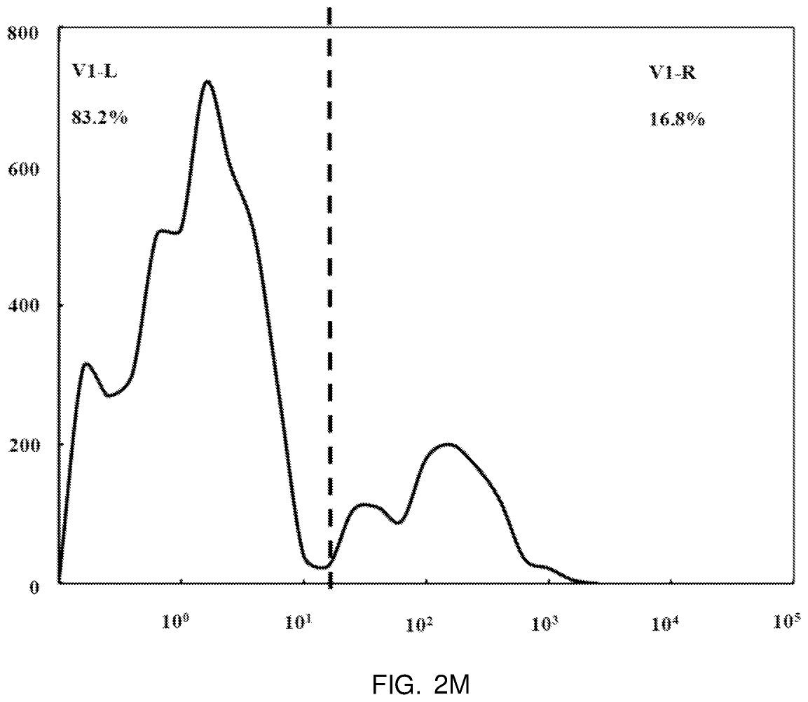

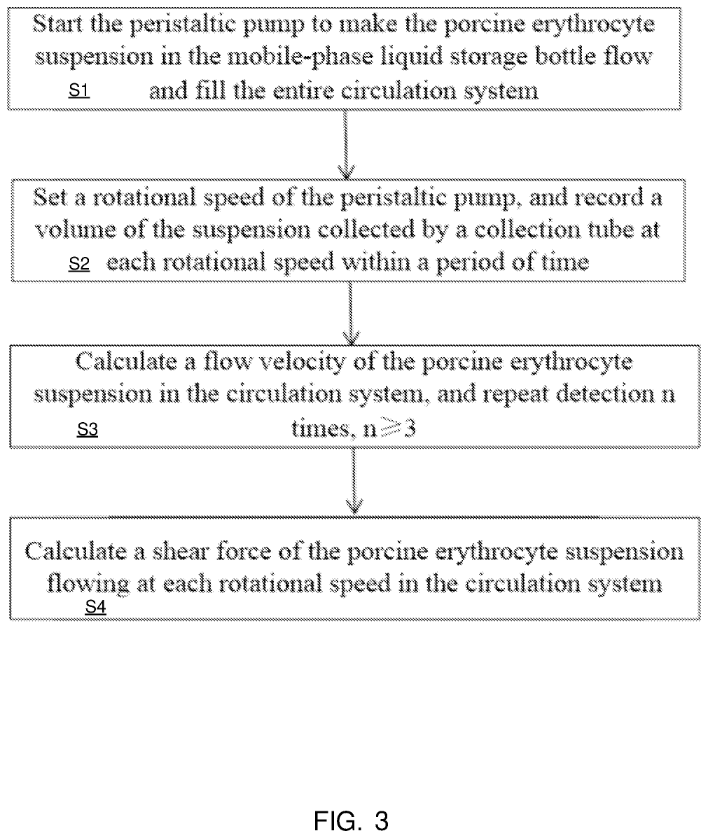

As shown in FIG. 3, the present disclosure further provides a circulation method for detecting immune adherence of porcine erythrocytes applied to the circulation system for detecting immune adherence of porcine erythrocytes. The method can include the following steps and the following steps were followed in an exemplary process to obtain the data seen in FIGS. 2A-2M:

S1, the peristaltic pump was started to make the porcine erythrocyte suspension in the mobile-phase liquid storage bottle flow and fill the entire circulation system;

S2, a rotational speed of the peristaltic pump was set, and a volume of the suspension collected by a collection tube at each rotational speed was recorded within 120 sec;

S3, a flow velocity of the porcine erythrocyte suspension in the circulation system was calculated, and detection was repeated 3 times; and

S4, a shear force of the porcine erythrocyte suspension flowing at each rotational speed in the circulation system was calculated.

In step S3, the flow velocity of the porcine erythrocyte suspension in the circulation system was calculated by formula 1:

$$Q = \frac{{V}_{n} - {V}_{0}}{120 s} \qquad ( 1 )$$

in the formula 1, Q represented the flow velocity, in mL/s; Vₙ represented the volume of the suspension collected at each rotational speed within the period of time, and V₀ represented an initial volume.

In step S4, the shear force of the porcine erythrocyte suspension flowing at each rotational speed in the circulation system was calculated by formula 2:

$$τ = \frac{6 μμ}{{a}^{2} b} \qquad ( 2 )$$

in the formula 2, τ was the shear force, in dynes/cm²; μ was a medium viscosity of 0.0076 P; a was a channel height of 0.013 cm of the flow chamber; b was a channel width of 1.0 cm; and Q was the flow velocity, in mL/s.

The calculation results of the flow velocity of the mobile phase and the flow shear force are shown in Table 1. It was seen that the flow velocity of the mobile phase in the circulation system was different under different rotational speeds of the peristaltic pump. Some studies believe that the shear force of venous blood flow in the natural state is (1-6) dynes/cm², and a shear force below 4 dynes/cm² under test conditions is a low shear force, which may lead to unstable flow of the mobile phase [65, 66]. Therefore, in this experiment, a rotational speed of the peristaltic pump was set to 4 rpm and the shear force was set to 5.3 dynes/cm² for subsequent experiments.

TABLE 1
Calculation results of mobile phase flow velocity and shear force

| Rotational | Initial | Final | | Flow | |
|---|---|---|---|---|---|
| speed | volume | volume | Time | velocity | |
| (rpm) | (mL) | (mL) | (S) | (mL/S) | τ (dynes/cm²) |
| 1 | 0 | 0.57 ± 0.047 | 120 | 0.0047 ± 0.0004 | 1.30 |
| 2 | 0 | 1.06 ± 0.047 | 120 | 0.00875 ± 0.0003 | 2.50 |
| 3 | 0 | 1.70 ± 0.081 | 120 | 0.0146 ± 0.0006 | 4.10 |
| 4 | 0 | 2.23 ± 0.047 | 120 | 0.0186 ± 0.0004 | 5.30 |
| 5 | 0 | 2.70 ± 0.081 | 120 | 0.0225 ± 0.0007 | 6.40 |
| 6 | 0 | 3.23 ± 0.120 | 120 | 0.027 ± 0.001 | 7.60 |
| 7 | 0 | 3.63 ± 0.047 | 120 | 0.0303 ± 0.0004 | 8.60 |
| 8 | 0 | 4.17 ± 0.047 | 120 | 0.0347 ± 0.0004 | 9.80 |
| 9 | 0 | 4.73 ± 0.047 | 120 | 0.0394 ± 0.0004 | 11.15 |
| 10 | 0 | 5.10 ± 0.081 | 120 | 0.0425 ± 0.0007 | 12.00 |
| 11 | 0 | 5.73 ± 0.047 | 120 | 0.0478 ± 0.0004 | 13.50 |
| 12 | 0 | 6.13 ± 0.047 | 120 | 0.0511 ± 0.0004 | 14.50 |
| 13 | 0 | 6.70 ± 0.081 | 120 | 0.0558 ± 0.0007 | 15.80 |
| 14 | 0 | 7.23 ± 0.081 | 120 | 0.0603 ± 0.004 | 17.10 |
| 15 | 0 | 7.67 ± 0.047 | 120 | 0.0638 ± 0.0004 | 18.10 |

Further, the method further included determining a maximum retention time phase:

after the porcine erythrocyte suspension flows and fills the entire circulation system, the rotational speed of the peristaltic pump was set;

a 0.2 mL sample of the porcine erythrocyte suspension was pipetted from the mobile-phase liquid storage bottle at regular intervals, and a surface fluorescence intensity of the porcine erythrocytes at each time point was detected by a flow cytometer, where 10⁵ of the porcine erythrocytes were detected in each tube and the samples at each time point were detected 3 times; and an average fluorescence intensity was calculated at each time point; and

differences between fluorescence intensities at different time points were calculated by one-way ANOVA.

Specifically, in the example, 20 mL of the porcine erythrocyte suspension of immune adherence-sensitized GFP-E. coli was injected into the liquid storage bottle; the liquid storage bottle, the peristaltic pump, and the flow chamber without PAMs were connected with sterile silicone hoses; a rotational speed of the peristaltic pump was set to 4 rpm, the peristaltic pump was started, and a timing test was started after the suspension was filled with the entire circulation system. A 0.2 mL sample of the porcine erythrocyte suspension was pipetted from the liquid storage bottle at 0 min, 5 min, 10 min, 15 min, 20 min, 25 min, 30 min, 35 min, 40 min, 45 min, 50 min, 55 min, and 60 min after the timing, and a surface fluorescence intensity of the porcine erythrocytes at each time point was detected by a flow cytometer, where 10⁵ of the porcine erythrocytes were detected in each tube and the samples at each time point were detected 3 times; and an average fluorescence intensity was calculated at each time point.

The experimental data were expressed as Mean±SD. One-way ANOVA was conducted on the experimental data using GraphPad Prism 5 software to compare the changes of fluorescence intensity at each time point. P<0.05 meant significant difference, P<0.01 meant extremely significant difference, and P>0.05 meant insignificant difference.

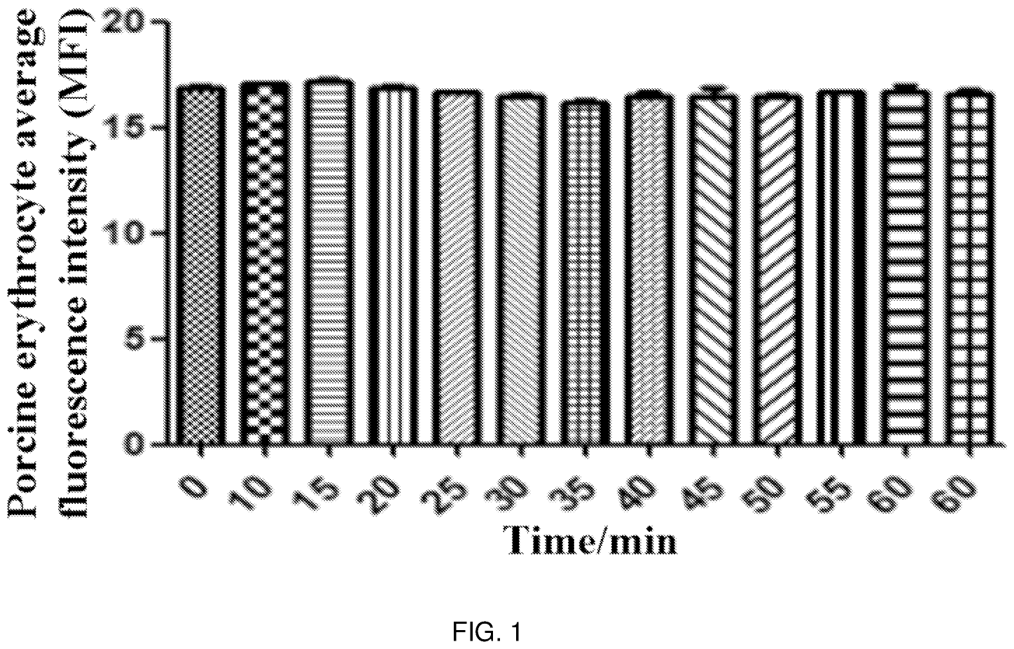

Detected by a flow cytometry, at each time point of 0 min, 5 min, 10 min, 15 min, 20 min, 25 min, 30 min, 35 min, 40 min, 45 min, 50 min, 55 min, and 60 min in the circulation, the average fluorescence intensities of porcine erythrocytes were 16.83±0.043, 17.03±0.23, 17.13±0.02, 16.83±0.02, 16.63±0.07, 16.47±0.06, 16.2±0.27, 16.43±0.084, 16.5±0.010, 16.5±0.05, 16.67±0.010, 16.7±0.10, and 16.6±0.20 (shown in Table 2). One-way ANOVA (as shown in FIG. 1) found that there was no significant difference between the fluorescence intensities at different time points (P>0.05). It was seen that during the circulation the porcine erythrocyte suspension in the circulation system, the fluorescence intensities of porcine erythrocytes did not change from 0 min to 60 min. This indicated that there was no loss of porcine erythrocyte-adherent GFP-E. coli in the circulation time of 60 min, meeting the needs of this experiment. FIG. 2A-M listed a representative flow chart.

TABLE 2
Detected values of surface fluorescence intensities
of porcine erythrocytes at each time point

| Time point | | | | Mean ± SD |
|---|---|---|---|---|
| 0 min | 17.00 | 16.90 | 16.90 | 16.83 ± 0.04 |
| 5 min | 17.10 | 17.10 | 16.90 | 17.03 ± 0.23 |
| 10 min | 17.30 | 17.20 | 16.90 | 17.13 ± 0.02 |
| 15 min | 17.00 | 16.80 | 16.90 | 16.83 ± 0.02 |
| 20 min | 16.60 | 16.57 | 16.72 | 16.63 ± 0.07 |
| 25 min | 16.70 | 16.50 | 16.70 | 16.47 ± 0.06 |
| 30 min | 16.10 | 16.20 | 16.30 | 16.20 ± 0.27 |
| 35 min | 16.30 | 16.90 | 16.10 | 16.43 ± 0.08 |
| 40 min | 15.90 | 16.60 | 17.00 | 16.50 ± 0.01 |
| 45 min | 16.50 | 16.70 | 16.30 | 16.50 ± 0.05 |
| 50 min | 16.66 | 16.67 | 16.68 | 16.67 ± 0.01 |
| 55 min | 17.10 | 16.70 | 16.30 | 16.70 ± 0.10 |
| 60 min | 16.70 | 16.80 | 16.30 | 16.60 ± 0.20 |

In summary, the present disclosure provides the following technical effects: in the circulation system and method for detecting immune adherence of porcine erythrocytes, the flow chamber, the gasket, the mobile-phase liquid storage bottle, the peristaltic pump, the silicone hoses, and the cell culture dish provided with a glass slide constitute a cyclic detection system, which can be used for determination of the mobile phase flow velocity, the shear force, and the maximum retention time. The present disclosure provides reliable working parameters for detecting immune adherence of porcine erythrocytes, which is convenient for technicians to operate and conduct follow-up research.

Specific examples are used herein to explain the principles and embodiments of the present disclosure. The foregoing description of the embodiments is merely intended to help understand the method of the present disclosure and its core ideas; besides, various modifications may be made by a person of ordinary skill in the art to specific embodiments and the scope of application in accordance with the ideas of the present disclosure. In conclusion, the content of the present description shall not be construed as limitations to the present disclosure.

What is claimed is:

1. A circulation system for detecting immune adherence of porcine erythrocytes, comprising: a flow chamber, a gasket, a mobile-phase liquid storage bottle, a peristaltic pump, silicone hoses, and a cell culture dish provided with a glass slide, wherein the gasket is placed on an edge of the glass slide, and the flow chamber is covered on the gasket; the flow chamber, the mobile-phase liquid storage bottle, and the peristaltic pump are connected in sequence with the silicone hoses to form the circulation system; and a porcine erythrocyte suspension of immune adherence-sensitized GFP-E. coli is injected into the mobile-phase liquid storage bottle.

2. The circulation system for detecting immune adherence of porcine erythrocytes according to claim 1, wherein 20 mL of the porcine erythrocyte suspension of immune adherence-sensitized GFP-E. coli is injected.

3. A circulation method for detecting immune adherence of porcine erythrocytes applied to the circulation system for detecting immune adherence of porcine erythrocytes according to claim 1, comprising the following steps:
S1, starting the peristaltic pump to make the porcine erythrocyte suspension in the mobile-phase liquid storage bottle flow and fill the circulation system;
S2, setting a rotational speed of the peristaltic pump, and recording a volume of the suspension collected by a collection tube at each rotational speed within a period of time;
S3, calculating a flow velocity of the porcine erythrocyte suspension in the circulation system, and repeating detection n times, n≥3; and
S4, calculating a shear force of the porcine erythrocyte suspension flowing at each rotational speed in the circulation system.

4. The circulation method for detecting immune adherence of porcine erythrocytes according to claim 3, wherein 20 mL of the porcine erythrocyte suspension of immune adherence-sensitized GFP-E. coli is injected.

5. The circulation method for detecting immune adherence of porcine erythrocytes according to claim 3, wherein in step S3, the flow velocity of the porcine erythrocyte suspension in the circulation system is calculated by formula 1:
$$Q = \frac{{V}_{n} - {V}_{0}}{120 s} \qquad ( 1 )$$
in the formula 1, Q represents the flow velocity, in mL/s; Vₙ represents the volume of the suspension collected at each rotational speed within the period of time; and V₀ represents an initial volume.

6. The circulation method for detecting immune adherence of porcine erythrocytes according to claim 4, wherein in step S3, the flow velocity of the porcine erythrocyte suspension in the circulation system is calculated by formula 1:
$$Q = \frac{{V}_{n} - {V}_{0}}{120 s} \qquad ( 1 )$$
in the formula 1, Q represents the flow velocity, in mL/s; Vₙ represents the volume of the suspension collected at each rotational speed within the period of time, and V₀ represents an initial volume.

7. The circulation method for detecting immune adherence of porcine erythrocytes according to claim 5, wherein in step S4, the shear force of the porcine erythrocyte suspension flowing at each rotational speed in the circulation system is calculated by formula 2:
$$τ = \frac{6 μμ}{{a}^{2} b} \qquad ( 2 )$$
in the formula 2, τ is the shear force, in dynes/cm²; μ is a medium viscosity of 0.0076 P; a is a channel height of 0.013 cm of the flow chamber; b is a channel width of 1.0 cm; and Q is the flow velocity, in mL/s.

8. The circulation method for detecting immune adherence of porcine erythrocytes according to claim 6, wherein in step S4, the shear force of the porcine erythrocyte suspension flowing at each rotational speed in the circulation system is calculated by formula 2:
$$τ = \frac{6 μμ}{{a}^{2} b} \qquad ( 2 )$$
in the formula 2, τ is the shear force, in dynes/cm²; μ is a medium viscosity of 0.0076 P; a is a channel height of 0.013 cm of the flow chamber; b is a channel width of 1.0 cm; and Q is the flow velocity, in mL/s.

9. The circulation method for detecting immune adherence of porcine erythrocytes according to claim 3, wherein the period of time is set to 120 sec.

10. The circulation method for detecting immune adherence of porcine erythrocytes according to claim 4, wherein the period of time is set to 120 sec.

11. The circulation method for detecting immune adherence of porcine erythrocytes according to claim 3, further comprising determining a maximum retention time of a mobile phase:
after the porcine erythrocyte suspension flows and fills the circulation system, setting the rotational speed of the peristaltic pump;
pipetting a 0.2 mL sample of the porcine erythrocyte suspension from the mobile-phase liquid storage bottle at regular intervals, and detecting a surface fluorescence intensity of the porcine erythrocytes at each time point by a flow cytometer, wherein 10⁵ of the porcine erythrocytes are detected in each tube and samples at each time point are detected 3 times; and calculating an average fluorescence intensity at each time point; and
calculating differences between fluorescence intensities at different time points by one-way ANOVA.

12. The circulation method for detecting immune adherence of porcine erythrocytes according to claim 5, further comprising determining a maximum retention time of a mobile phase:
after the porcine erythrocyte suspension flows and fills the circulation system, setting the rotational speed of the peristaltic pump;
pipetting a 0.2 mL sample of the porcine erythrocyte suspension from the mobile-phase liquid storage bottle at regular intervals, and detecting a surface fluorescence intensity of the porcine erythrocytes at each time point by a flow cytometer, wherein 10⁵ of the porcine erythrocytes are detected in each tube and samples at each time point are detected 3 times; and calculating an average fluorescence intensity at each time point; and
calculating differences between fluorescence intensities at different time points by one-way ANOVA.
